(19) United States
(12) Patent Application Publication
(10) Pub. No.: US 2015/0018619 A1
(43) Pub. Date: Jan. 15, 2015

(54) SPACE-SAVING FLAT INTERCONNECTION

(71) Applicant: GYRUS ACMI INC. (d.b.a. Olympus Surgical Technologies America), Southborough, MA (US)

(72) Inventors: Doron Adler, Haifa (IL); Arie Blumenzweig, Netanya (IL)

(21) Appl. No.: 14/179,577

(22) Filed: Feb. 13, 2014

Related U.S. Application Data
(63) Continuation-in-part of application No. 13/940,282, filed on Jul. 12, 2013.

(51) Int. Cl. H01L 27/146; A61B 1/05

(52) U.S. Cl. CPC H01L 27/14636; H01L 27/14687; A61B 1/05
USPC 600/112; 257/448; 438/66

(57) ABSTRACT

Apparatus, consisting of an integrated circuit (IC) die, and a circuit. The IC die includes a semiconductor substrate having a substrate plane face and a substrate edge, an imaging array formed on the substrate plane face, and a plurality of array pads mounted on the substrate plane face and connected to the imaging array. The circuit includes a circuit substrate having a circuit face and a circuit edge butted to the substrate edge, a plurality of circuit pads mounted on the circuit face, and a plurality of traces mounted on the circuit face and connected to the circuit pads. The apparatus further includes a plurality of connectors coupling the array pads to the circuit pads.

### CROSS-REFERENCE TO RELATED APPLICATIONS

This application is a continuation-in-part of U.S. patent application Ser. No. 13/940,282, entitled “Endoscope Small Imaging System,” which is incorporated herein by reference.

### FIELD OF THE INVENTION

The present invention relates generally to imaging, and specifically to a connection for an imaging system of an endoscope having a small diameter.

### BACKGROUND OF THE INVENTION

U.S. Patent Application 2005/0267328, to Blumzvig et al., whose disclosure is incorporated herein by reference, describes a miniature camera head. The head includes an electronic imaging device having an optical objective and an image sensor that is non-perpendicular to an optical axis of the objective.

U.S. Patent Application 2013/0129334, to Wang et al., whose disclosure is incorporated herein by reference, describes a capsule camera with on-board storage. The camera integrates an image sensor, a light source, and at least one system processing chip.

U.S. Pat. No. 6,900,527, to Miks et al., whose disclosure is incorporated herein by reference, describes a lead-frame method and assembly for interconnecting circuits within a circuit module. The system is stated to allow the circuit module to be fabricated without a circuit board substrate.

U.S. Pat. No. 7,019,387, to Miks et al., whose disclosure is incorporated herein by reference, describes a lead-frame connector and circuit module assembly. The connector and assembly are stated to allow fabrication of a pin-connector type circuit module without a circuit board substrate and discrete connector.

U.S. Pat. No. 8,263,438, to Seah et al., whose disclosure is incorporated herein by reference, describes a method for connecting a die assembly to a substrate in an integrated circuit. The die assembly and the substrate are arranged to be electrically connected through one or more routing circuits carried on a flexible strip.

U.S. Pat. No. 8,438,730, to Ciminelli, whose disclosure is incorporated herein by reference, describes a method of assembling an inkjet printhead. The method includes affixing a printhead die to a die location portion of a support surface, and affixing a portion of an attachment surface of a flexible circuit to the support surface adjacent to the die location portion of the support surface.

U.S. Patent Application 2010/0283818, to Bruce et al., whose disclosure is incorporated herein by reference, describes a printhead assembly for an inkjet-printing device that includes a printhead die and a flexible circuit connected to the printhead die.

U.S. Pat. No. 7,804,985, to Szewerenko et al., whose disclosure is incorporated herein by reference, describes impact resistant circuit modules for enclosing a die having a sensor area. The modules include a flexible circuit and a die coupled thereto.

U.S. Patent Application 2011/0210441, to Lee et al., whose disclosure is incorporated herein by reference, describes a chip package that includes a semiconductor chip, a flexible circuit film and a substrate. The substrate has a circuit structure in the substrate. The flexible circuit film is connected to the circuit structure of the substrate through metal joints, an anisotropic conductive film or wirebonding wires.

U.S. Patent Application 2012/0274705, to Petersen et al., whose disclosure is incorporated herein by reference, describes a printhead assembly. The assembly includes a printhead die, a base coupled to the printhead die, a flexible circuit mounted on the base and electrically connected to the printhead, and an adhesive sandwiched between the base and the flexible circuit.

U.S. Pat. No. 8,179,428, to Minami et al., whose disclosure is incorporated herein by reference, describes an imaging apparatus for an electronic endoscope which uses a “bare chip” of a CCD (charge coupled device) together with a circuit board having approximately the same thickness as the bare chip.

U.S. Pat. No. 6,659,940, to Adler, whose disclosure is incorporated herein by reference, describes an endoscope having restricted dimensions. The endoscope has an image “gatherer,” an image distorter, and an image sensor shaped to fit within the restricted dimensions.

U.S. Pat. No. 4,684,222, to Borelli et al., whose disclosure is incorporated herein by reference, describes a method for producing small lenses which may be formed to be anamorphic.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

### SUMMARY OF THE INVENTION

An embodiment of the present invention provides apparatus, including:

an integrated circuit (IC) die, having:

a semiconductor substrate having a substrate plane face and a substrate edge;

an imaging array formed on the substrate plane face; and

a plurality of array pads mounted on the substrate plane face and connected to the imaging array;

a circuit, having:

a circuit substrate having a circuit face and a circuit edge butted to the substrate edge;

a plurality of circuit pads mounted on the circuit face; and

a plurality of traces mounted on the circuit face and connected to the circuit pads; and

a plurality of connectors coupling the array pads to the circuit pads.

Typically, the circuit includes a flexible circuit.

The circuit substrate may be formed from a dielectric.

In a disclosed embodiment the apparatus further includes cement fixedly holding the circuit edge to the substrate plane edge. The cement may also hold the connectors in place.

In a further disclosed embodiment the IC die includes a multiplicity of additional array pads mounted on the substrate plane face and connected to the imaging array, the apparatus further including:

an additional circuit, having:

an additional circuit substrate having an additional circuit face and an additional circuit edge separated from the substrate plane edge by a preset distance;

a multiplicity of additional circuit pads mounted on the additional circuit face; and

a multiplicity of additional traces mounted on the additional circuit face and connected to the additional circuit pads; and

a multiplicity of additional connectors coupling the additional array pads to the additional circuit pads.

Typically, the preset distance is greater than or equal to a length of the circuit pads.

The additional circuit may be fixedly attached to the circuit.

In an embodiment the IC die, the circuit, and the plurality of connectors are located in a distal end of an endoscope.

There is further provided, according to an embodiment of the present invention, an endoscope camera, including:

a cylindrical enclosure having an enclosure diameter;

an integrated circuit (IC) die mounted within the enclosure, the die having:

a semiconductor substrate having a substrate plane face and a substrate edge;

an imaging array formed on the substrate plane face; and

a plurality of array pads mounted on the substrate plane face and connected to the imaging array;

a right-angle transparent prism having a rectangular entrance face, an exit face, and an hypotenuse configured to reflect radiation from the entrance face to the exit face, the entrance face having a first edge longer than a second edge, the prism being mounted within the enclosure so that the first edge is parallel to the enclosure diameter and so that the exit face mates with the substrate plane face;

a circuit, including:

a circuit substrate having a circuit face and a circuit edge butted to the substrate edge;

a plurality of circuit pads mounted on the circuit face; and

a plurality of traces mounted on the circuit face and connected to the circuit pads;

a plurality of connectors coupling the array pads to the circuit pads; and

optics, configured to receive incoming radiation from an object, mounted so as to transmit the incoming radiation to the imaging array via the entrance and exit faces of the prism.

There is further provided, according to an embodiment of the present invention, a method, including:

providing an integrated circuit (IC) die consisting of a semiconductor substrate having a substrate plane face and a substrate edge;

forming an imaging array on the substrate plane face;

mounting a plurality of array pads on the substrate plane face and connecting the pads to the imaging array;

providing a circuit consisting of a circuit substrate having a circuit face and a circuit edge;

butting the circuit edge to the substrate edge;

mounting a plurality of circuit pads on the circuit face;

mounting a plurality of traces on the circuit face and connecting the traces to the circuit pads; and

coupling a plurality of connectors between the array pads and the circuit pads.

The present invention will be more fully understood from the following detailed description of the embodiments thereof, taken together with the drawings in which:

### BRIEF DESCRIPTION OF THE DRAWINGS

FIG. 1 is a schematic illustration of an endoscopic imaging system, according to an embodiment of the present invention;

FIG. 2A is a schematic perspective illustration of a camera of the imaging system, according to an embodiment of the present invention;

FIG. 2B and FIG. 2C are schematic sectional views of the camera, according to an embodiment of the present invention;

FIG. 2D is a schematic perspective view of an element of the camera, according to an embodiment of the present invention;

FIG. 2E is a schematic top view of elements of the camera, according to an embodiment of the present invention;

FIG. 3A is a schematic sectional view of an alternative camera, and FIG. 3B is a schematic top view of elements of the alternative camera, according to an embodiment of the present invention;

FIG. 4 is a schematic conceptual representation of the operation of optics of the camera of FIGS. 2A-2E, according to an embodiment of the present invention;

FIG. 5 is a flowchart describing steps in operation of the imaging system, according to an embodiment of the present invention;

FIG. 6 is a schematic top view of a camera, and FIG. 7 is a schematic sectional view of the camera, according to a further alternative embodiment of the present invention; and

FIG. 8 is a schematic top view of a camera, and FIG. 9 is a schematic sectional view of the camera according to a yet further embodiment of the present invention.

### DETAILED DESCRIPTION OF EMBODIMENTS OVERVIEW

Endoscopes used in surgery preferably have small dimensions. Especially for minimally invasive surgery, the smaller the dimensions of the endoscope and its elements, the less the trauma on patients undergoing the surgery. A system which enables a reduction in diameter of the endoscope, without a concomitant reduction in efficiency of operation of the endoscope, would be advantageous.

An embodiment of the present invention provides a connection between an integrated circuit (IC) die and a circuit which is typically a flexible circuit. Both the die and the circuit are typically incorporated into a camera located at the distal end of an endoscope. The connection is substantially planar, and so, when implemented in the endoscope, allows dimensions of the endoscope to be minimized.

The IC die comprises a semiconductor substrate upon which are formed an imaging array and a plurality of conductive array pads which are connected to the array. The circuit also comprises a substrate, typically a dielectric substrate, upon which are formed a plurality of conductive traces which are connected to respective conductive circuit pads on the substrate. Both the die and the circuit are typically planar, with a thickness which is of the order of 100 microns or less. The circuit and the die are connected together by butting an edge of the die to an edge of the circuit, and coupling the circuit pads to the array pads with a plurality of connectors. Typically the butted edges of the circuit and die are cemented together to increase the mechanical stability of the connection.

By butting the edges of the circuit and IC die, rather than overlaying one over the other, or using a support for both the circuit and die, the dimensions of the connection between the circuit and the die are minimized. Thus, an endoscope camera using the connection requires less space than alternative systems that are used in the prior art.

### DETAILED DESCRIPTION

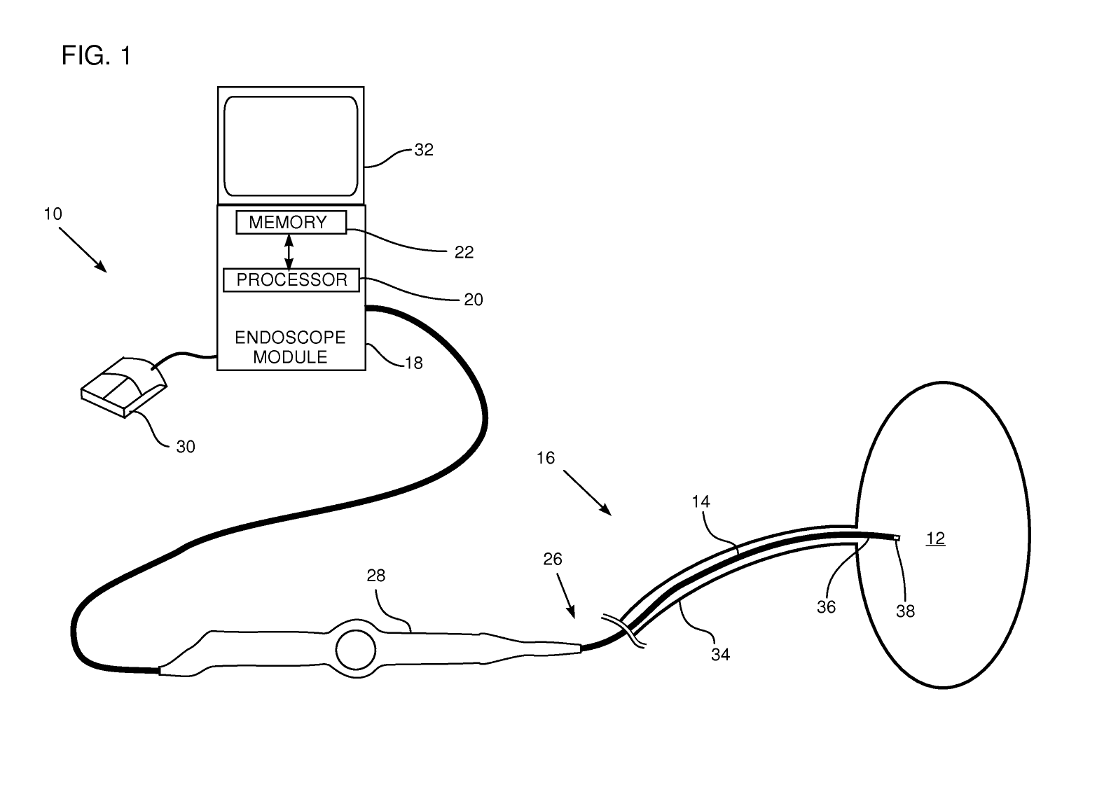

Reference is now made to FIG. 1, which is a schematic illustration of an endoscopic imaging system 10, according to an embodiment of the present invention. System 10 may be used in an invasive medical procedure, typically a minimally invasive procedure, on a body cavity 12 of a human patient in order to image part or all of the body cavity. By way of example, in the present description the body cavity is assumed to be the bladder of a patient, and body cavity 12 is also referred to herein as bladder 12. However, it will be understood that system 10 may be used to image substantially any human body cavity, such as the gastrointestinal organs, the bronchium, or the chest, or a non-human cavity.

System 10 comprises an imaging apparatus 14 which enables delivery of an endoscope 16 to bladder 12. Apparatus 14 is typically in the form of a tube which is able to traverse a lumen of a patient's body, so that apparatus 14 is also referred to herein as tube 14. Endoscope 16 is controlled by an endoscope module 18 having a processor 20 communicating with a memory 22. Apparatus 14 is connected at its proximal end 26 to a handle 28 which enables an operator, herein assumed to be a physician, of system 10 to insert the apparatus into the bladder as well as to manipulate the endoscope so as to acquire images of the bladder. In some embodiments of the present invention, rather than manual manipulation of endoscope 16 using handle 28, the endoscope is manipulated automatically, such as by scanning, so as to acquire its images.

The operator is able to provide input to module 18 via controls 30, which typically comprise at least one of a keyboard, a pointing device, or a touch screen. Alternatively or additionally, at least some of controls 30 may be incorporated into handle 28. For simplicity, controls 30 are herein assumed to comprise a mouse, so that the controls are also referred to herein as mouse 30.

The processor uses software, typically stored in memory 22, to control system 10. Results of the actions performed by processor 20 may be presented on a screen 32 to the operator of system 10, the screen typically displaying an image of bladder 12 that is generated by system 10. The image displayed on screen 32 is assumed to be rectangular, and to have a display aspect ratio (DAR) of s:1, where DAR is the ratio of the image width to the image height. Typically, although not necessarily, the DAR of the image corresponds to the physical dimensions of screen 32, and the image DAR may be one of the standard ratios known in the art, such as 4:3. A difference between the DAR of the image and the dimensions of the screen may be accommodated by incorporating black “bands” on the screen, as is done in projecting high definition images with an aspect ratio of 16:9 onto a screen with width:height dimensions 4:3. As is explained in more detail below, embodiments of the present invention are able to present undistorted images of an object viewed by system 10 on screen 32 for substantially any desired value of DAR.

By way of example, in the following description, except where otherwise indicated, DAR of screen 32 is assumed to be 4:3, and the image formed on screen 32 is assumed to be in a format of 768 pixels wide×576 pixels high.

The software for operating system 10 may be downloaded to processor 20 in electronic form, over a network, for example, or it may, alternatively or additionally, be provided and/or stored on non-transitory tangible media, such as magnetic, optical, or electronic memory.

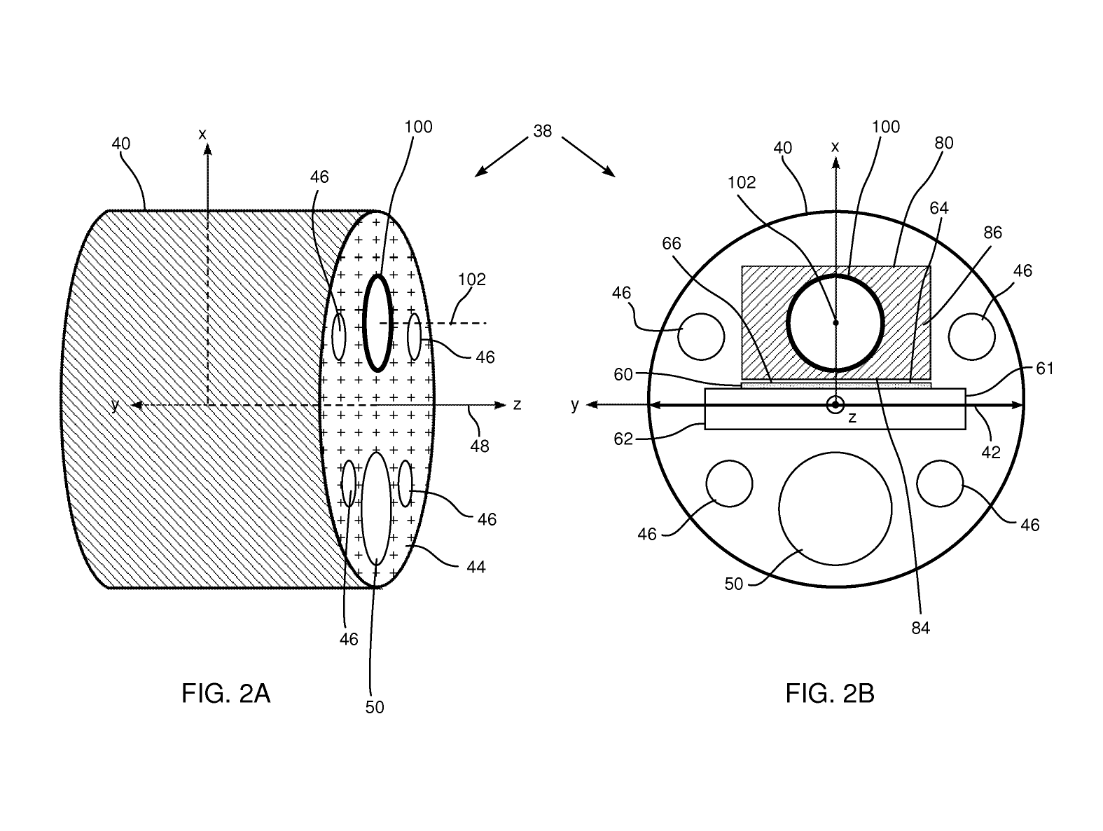

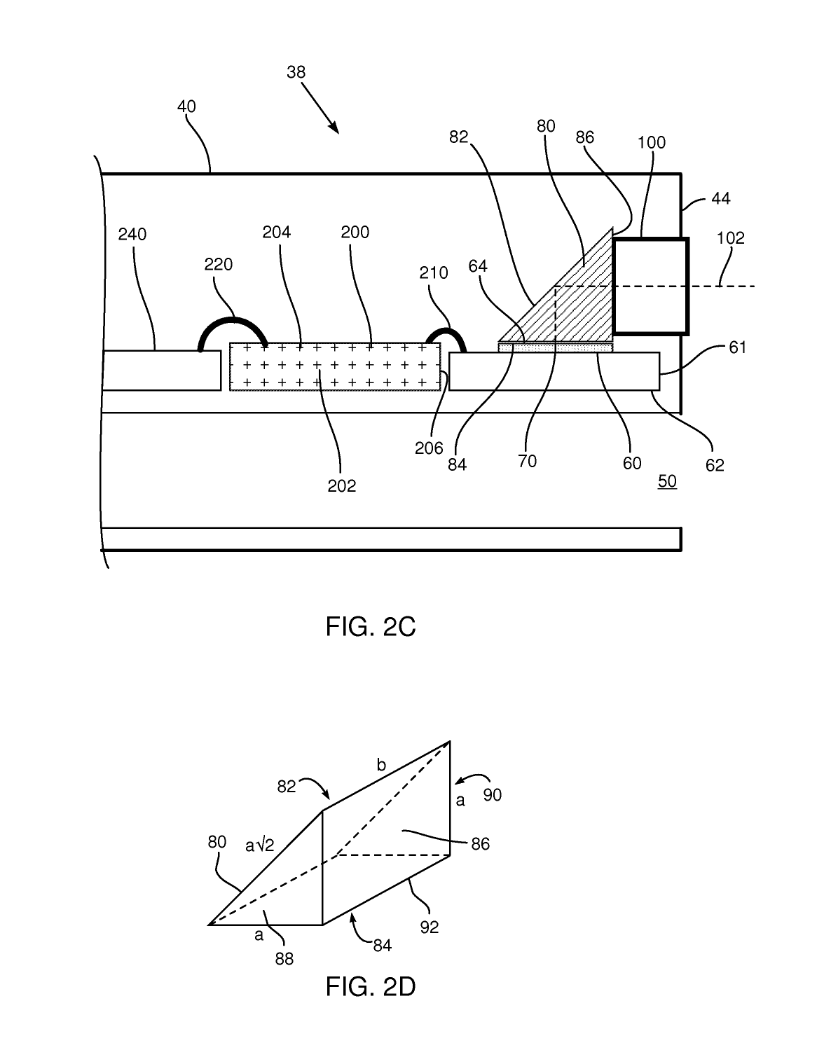

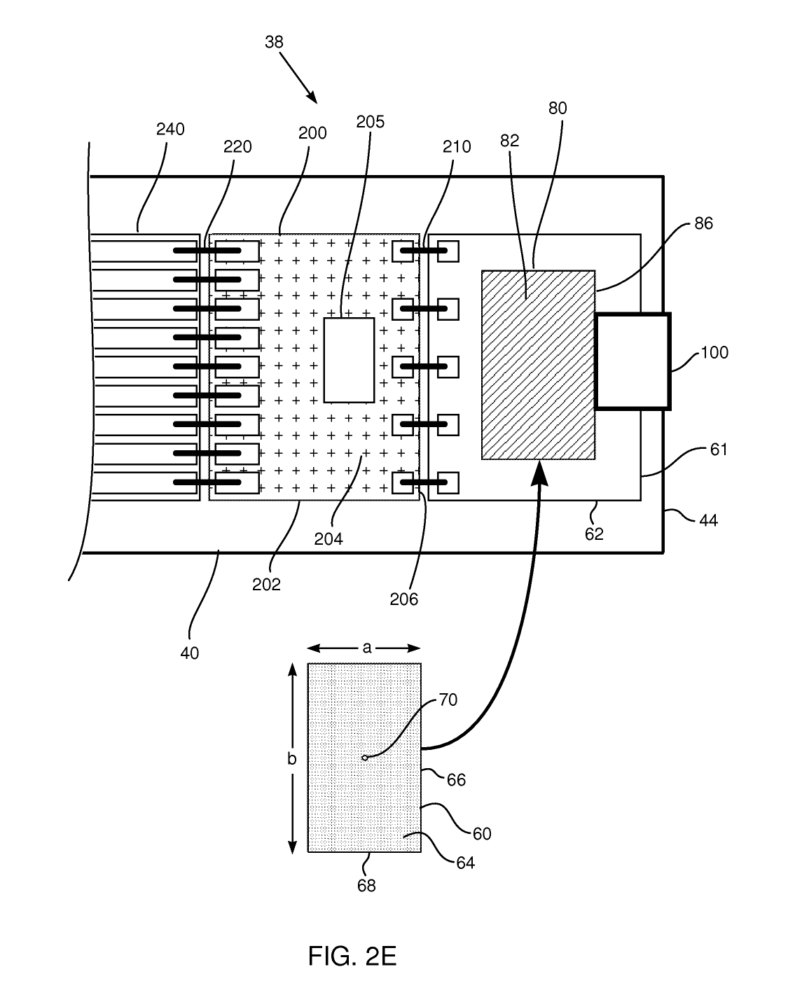

To operate system 10, the physician inserts tube 14 through a urethra 34 until a distal end 36 of the tube enters the bladder. Distal end 36 of tube 14 comprises a camera 38. The structure and operation of camera 38 are described below with reference to FIGS. 2A-2E.

FIG. 2A is a schematic perspective view of camera 38, FIGS. 2B and 2C are schematic sectional views of the camera; FIG. 2D is a schematic perspective view of an element of the camera, and FIG. 2E is a schematic top view of elements of the camera, according to an embodiment of the present invention. Camera 38 comprises a cylindrical enclosure 40, having an internal enclosure diameter 42, the cylindrical enclosure being terminated at its distal end by an approximately plane surface 44. Typically, cylindrical enclosure 40 is integral with tube 14. For clarity in the description of camera 38, cylindrical enclosure 40 is assumed to define a set of xyz orthogonal axes, with the z axis corresponding to a symmetry axis 48 of the cylindrical enclosure, and the x axis in the plane of the paper in FIG. 2A. FIG. 2B is a schematic sectional view of camera 38 in an xy plane, the view being drawn with plane surface 44 removed.

Camera 38 comprises four generally similar light channels 46, which traverse tube 14 and which, at the distal end of the tube, are approximately parallel to the z axis. Channels 46 exit surface 44, and the channels are typically tubes which contain fiber optics (not shown in the diagram) for transmitting light that exits from surface 44. The light from the fiber optics illuminates elements of cavity 12, and returning light from the illuminated elements is used by camera 38 to generate an image of the elements, as described below. Alternatively, in some embodiments light channels 46 are fiber optics.

Camera 38 also comprises a working channel 50 which traverses tube 14, and which, at the distal end of the tube, is approximately parallel to the z axis. Working channel 50 is typically larger than light channels 46, and may be used by the physician to insert a variety of surgical tools, such as a biopsy tool, into cavity 12.

Camera 38 generates its images in a rectangular array 60 of imaging pixels, the array being formed as part of an integrated circuit die 61 that is mounted within enclosure 40. Die 61 is assumed to be in the shape of a parallelepiped having a semiconductor substrate 62, and the rectangular array is typically a charge coupled device (CCD) that is formed on a planar surface of substrate 62. The substrate acts as a support for the array. Array 60 has a face 64 which receives radiation forming the images generated by the array. The array has two edges, a first edge 66 having a length “b,” and a second edge 68 having a length “a.”

In embodiments of the present invention the two edges of array 60 are unequal in length, i.e., a≠b, and for clarity in the disclosure, edge 66 is assumed to be longer than edge 68, i.e., b>a, so that edge 66 may also be referred to as the longer edge or the width, and edge 68 may also be referred to as the shorter edge or the height. Array 60 has an array aspect ratio (AAR) of b:a, and if the pixels of array 60 are square, then a pixel aspect ratio (PAR) of array 60, corresponding to the ratio of the number of pixels in a row to the number of pixels in a column, is also b:a. Rectangular array 60 has a center of symmetry 70.

In a disclosed embodiment array 60 has b=500 μm and a=280 μm, and the array is formed of 2.5 μm square pixels. In this case the pixel dimensions of the array are 200×112, and AAR=PAR=500:280=200:112. Arrays with dimensions similar to these are known in the art, and may be supplied by imaging sensor providers, such as Forza Silicon Corporation, of Pasadena, Calif.

Die 61 is mounted within enclosure 40 so that axis 48 of the enclosure is parallel to face 64 of the rectangular array, and so that the longer edge of the array is parallel to diameter 42.

As shown in FIGS. 2C and 2D, a right-angle transparent prism 80 is mounted within enclosure 40. Prism 80 has three rectangular faces: a hypotenuse face 82, a base face 84, also herein termed exit face 84, and an upright face 86, also herein termed entrance face 86. The prism also has a first isosceles right-angle triangle face 88 and a second isosceles right-angle triangle face 90. The dimensions of prism 80 are implemented so that exit face 84 has the same dimensions as array 60, i.e., the exit face is a rectangle having edge lengths a and b. Entrance face 86 has the same dimensions as exit face 84, i.e., the entrance face is a rectangle having edge lengths a and b. Entrance face 86 and exit face 84 have a common edge 92 with length a, i.e., the common edge is a longer edge of the exit and entrance faces.

The lengths of the sides forming the right angle of isosceles right-angle triangle face 88, and of isosceles right-angle triangle face 90, correspond to the length of the shorter edge of array 60, so that the two isosceles triangles of faces 88, 90, have lengths: a, a, a\sqrt{2}. Rectangular hypotenuse face 82 has edge lengths a\sqrt{2}, b.

Prism 80 is mounted onto array 60 so that exit face 84 mates with the array, i.e., so that the shorter edge of the exit face aligns with the shorter edge of the array, and so that the longer edge of the exit face aligns with the longer edge of the array. The mounting of the prism onto the array may be implemented using an optical adhesive, typically an epoxy resin, that cements the prism to the array. Such a mounting reduces undesired reflections from the exit face of the prism, as well as from face 64 of the array.

Optical elements 100, herein termed optics 100, are mounted within enclosure 40 so that they align with the entrance face of prism 80. Typically, optics 100 are cylindrical as illustrated in the figures. Typically, the mounting comprises cementing optics 100 to entrance face 86 using an optical adhesive. Optics 100 have an optic axis 102, and the optics are mounted so that the optic axis, after reflection in hypotenuse 82, intersects center 70 of array 60.

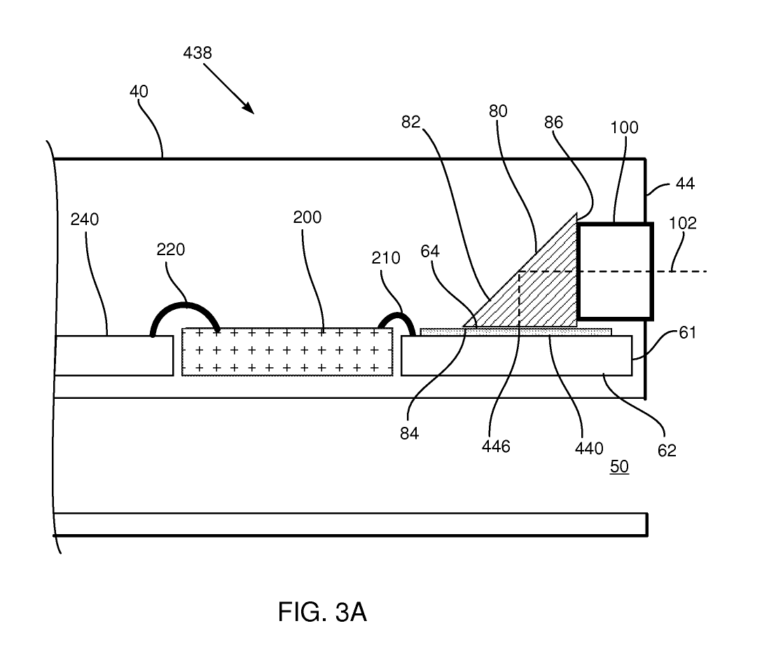

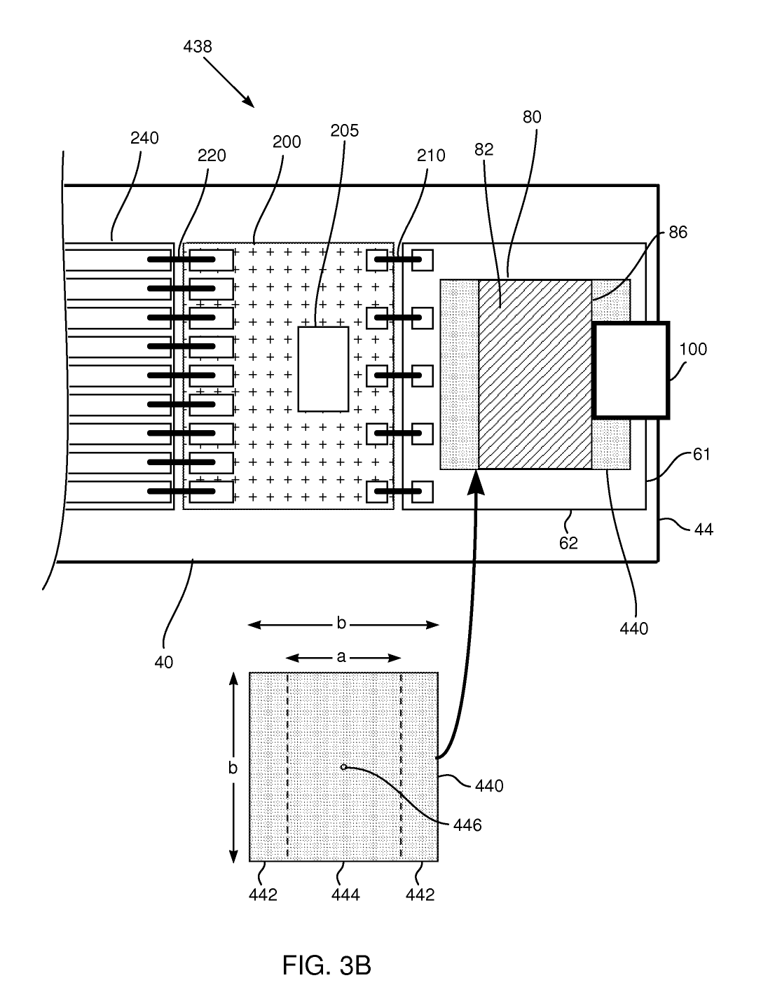

FIG. 3A is a schematic sectional view of a camera 438, and FIG. 3B is a schematic top view of elements of the camera, according to an alternative embodiment of the present invention. Apart from the differences described below, the operation of camera 438 is generally similar to that of camera 38 (FIGS. 2A-2E), and elements indicated by the same reference numerals in both cameras 38 and 438 are generally similar in construction and in operation.

In contrast to camera 38, which uses rectangular array 60 having unequal edges, camera 438 uses a square array 440. Square array 440 is configured to have its edge equal in length to the longer side of exit face 84, i.e., array 440 has an edge length b.

Prism 80 is mounted onto array 440 so that the shorter edges of the exit face align with the edges of the array. The mounting is typically symmetrical, so that as illustrated in FIG. 3B, there are approximately equal sections 442 which do not receive radiation from the exit face, and a rectangular section 444, having dimensions of b x a, which aligns with and is cemented to the exit face so as to receive radiation from the face. Optics 100 are mounted so that optic axis 102, after reflection in hypotenuse 82, intersects a center of symmetry 446 of section 444.

In a disclosed embodiment array 440 has b=500 μm and the array is formed of 2.5 μm square pixels. In this case the pixel dimensions of the array are 200×200. (Arrays with dimensions similar to these are also known in the art, and may be supplied by imaging sensor providers, such as the provider referred to above.) In the disclosed embodiment section 444 has dimensions of 500 μm×280 μm and pixel dimensions of 200×112, corresponding to the parameters of camera 38.

When camera 438 operates, section 444 is an active region of array 440, acquiring images projected onto the section via the completely filled exit face of the prism, whereas sections 442 are inactive regions.

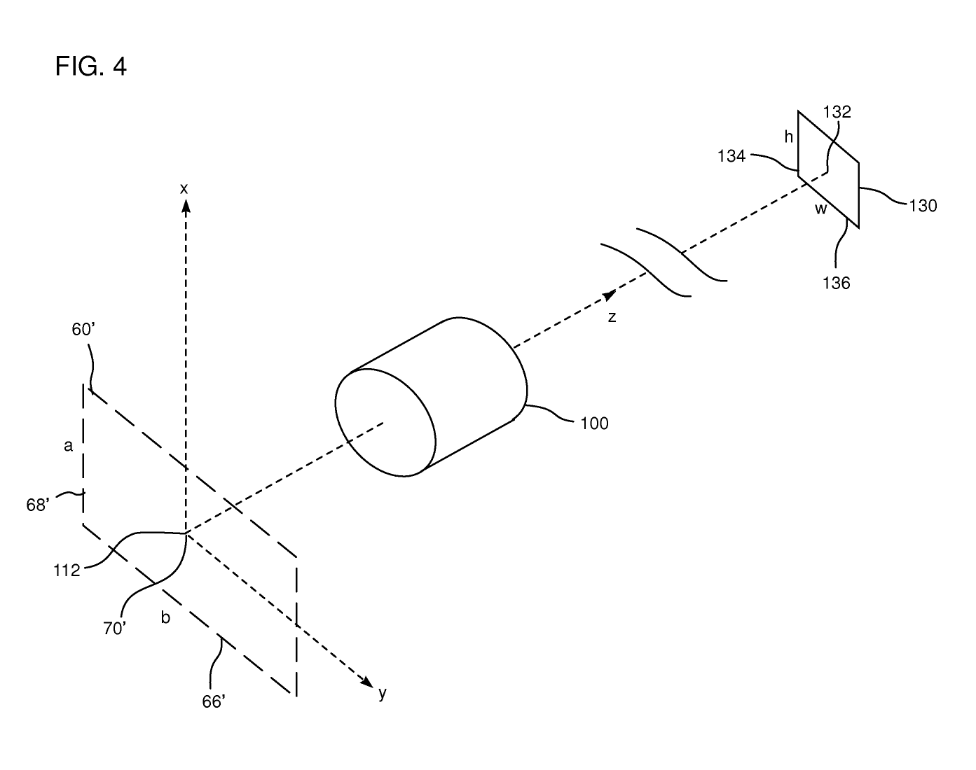

FIG. 4 is a schematic conceptual representation of the operation of optics 100, according to an embodiment of the present invention. The figure has been drawn using the set of xyz axes defined above for FIGS. 2A-2E, and assumes that camera 38 is being considered. Those having ordinary skill in the art will be able to adapt the following description for the case of camera 438. For simplicity, the figure has been drawn without the presence of prism 80, so that array 60 with center 70 is represented by a congruent array 60′ with a center 70′. Array 60′ has edges 66′ and 68′, corresponding to edges 66 and 68 of array 60. Edges 66′ and 68′ are parallel to the y and x axes, and center 70′ is on the z axis. The following description reverts to referring to array 60 with center 70.

From a conceptual point of view, optics 100 may be considered to have the properties of an anamorphic lens, having different magnifications in the x direction and in the y direction. For simplicity the following description assumes that an object 130 is very distant from optics 100, so that the object is effectively at infinity, and those having ordinary skill in the art will be able to adapt the description for objects that are closer to optics 100. Furthermore, object 130 is assumed to be rectangular, with a center 132 on the z axis and edges 134 and 136 respectively parallel to the x and y axes. Edge 134 has a height h and edge 136 has a width w, giving an object aspect ratio of w:h.

Optics 100 is assumed to focus rays from center point 132 of object 130 to a focal point 112 on the z axis, and the optics are positioned so that center 70 of array 60 coincides with focal point 112. Object 130 may thus be completely in focus on array 60.

Typically, optics 100 are configured so that the image of object 130 completely fills the exit face of the prism and completely covers array 60; this configuration utilizes all the pixels of array 60. However, except for the case where w:h=b:a, the complete coverage entails optics 100 distorting the image of object 130, so that the image produced by the optics is no longer geometrically similar to the object. The distortion introduced by the optics is equivalent to the optics behaving as an anamorphic system, i.e., generating magnifications of the image on the array which are different in the x direction and in the y direction.

The magnifications for optics 100 are given by the following equations:

$${m}_{x} = \frac{a}{h} ; {m}_{y} = \frac{b}{w} \qquad ( 1 )$$

where mₓ is a height magnification of the optics, in the x direction, and

m_{y} is a width magnification of the optics, in the y direction.

A measure of the distortion produced by the optics is given by the ratio of the width:height magnifications in the two directions, i.e., a ratio of the width magnification m_{y} to the height magnification mₓ:

$$D = \frac{{m}_{y}}{{m}_{x}} = \frac{bh}{aw} \qquad ( 2 )$$

where D is a distortion metric for optics 100, equal to the ratio of the width:height magnifications.

As a first numerical example of the distortion introduced by optics 100, assume that object 130 has dimensions of w=4000 μm and h=3000 μm so that the object has an aspect ratio of 4:3. This aspect ratio is a typical value for “standard” imaging optics. Assume further that array 60 has the dimensions of the disclosed embodiment above, i.e., a width of 500 μm and a height of 280 μm. In this case, from equation (1), optics 100 are configured to have the following magnifications:

$${m}_{x} = \frac{280}{3000} = 0.093 ; {m}_{y} = \frac{500}{4000} = 0.125 \qquad ( 3 )$$

From equation (2), the ratio of the width:height magnifications, the distortion D, of optics 100 in this case is:

$$D = \frac{bh}{aw} = \frac{500 \cdot 3000}{280 \cdot 4000} = 1.34 \qquad ( 4 )$$

As a second numerical example, assume that object 130 is square, so that w=h, corresponding to an aspect ratio of 1:1. In this case the distortion D introduced by optics 100, from equation (2), is equal to the aspect ratio of array 60, i.e., for the disclosed embodiment above,

$$D = \frac{b}{a} = \frac{500}{280} = 1.79 \qquad ( 5 )$$

As a third numerical example, assume that object 130 has an aspect ratio of b:a, equal to the aspect ratio of array 60. In this case there is no distortion introduced by optics 100, i.e., the magnifications in the x and y directions are equal, mₓ=m_{y}, and D=1.

The description of optics 100 above has referred to the height and width magnifications, mₓ, m_{y} in the x and y directions, required by the optics in order to image object 130 onto array 60. For each specific magnification, there is a corresponding focal length fₓ, f_{y} of optics 100. An approximation for the focal lengths may be determined from equation (6) for a simple lens:

$$f = \frac{{md}_{o}}{m + 1} \qquad ( 6 )$$

where f is a required focal length of optics 100, d_{O} is the distance from the optics to object 130, and m is a desired magnification.

Those having ordinary skill in the art will be able to use equation (6), or other equations well known in the optical arts, in order to calculate focal length fₓ, f_{y} of optics 100, and to calculate other parameters for the optics and for system 10.

Optics 100 may be implemented using individual “conventional” components or lenses, or even as a single lens, by methods which are well-known in the art. For example, U.S. Pat. No. 4,684,222, referenced above, describes a method for producing small anamorphic lenses. Alternatively or additionally, optics 100 may be implemented using gradient-index (GRIN) optics, using methods known in the art. Using GRIN optics allows a face of optics 100 that is to mate with prism entrance face 86 to be made plane, facilitating the cementing of the optics to the entrance face. In addition, GRIN optics may reduce the size of optics 100 compared to the size required by conventional components.

Returning to FIGS. 2C and 2E, circuitry 200, which is typically implemented as an integrated circuit, generates clocking signals which drive array 60 and which are provided to the array by connectors 210. Circuitry 200 is typically formed on a parallelepiped substrate 202, and the circuitry includes and is driven by a local processor 205, typically formed on a circuit surface 204 of substrate 202. Processor 205 has overall control of the operation of the circuitry. As is illustrated in FIGS. 2C and 2E, an edge 206 of substrate 202 butts with an edge of substrate 62. Signals generated by the array in response to radiation incident on the array are transferred by connectors 210 to circuitry 200. The signals generated by array 60 are initially in analog form, and circuitry 200, inter alia, amplifies and digitizes the analog signals, typically to form frames of digital images, corresponding to the optical images incident on array 60. Circuitry 200 then transfers the digitized frames to processor (FIG. 1) via conducting elements 220. At least some of elements 220 are typically formed on, or are connected to, a flexible printed circuit board 240 which is installed in tube 14. However, any other method known in the art, such as using fibre optics and/or a wireless transmitter, may be implemented to transfer data generated by circuitry 200 to processor 20.

The digitized images output from array 60 have been optically distorted by optics 100 according to the distortion metric D, defined above with respect to equation (2). In order to display the images acquired by array 60 in an undistorted manner on screen 32, circuitry 200 applies a numerical “un-distortion” factor U to the received digitized images, so that the digitized images received by processor 20 are in an undistorted format. Alternatively, the un-distortion factor U may be applied by processor 20 to the distorted digitized images output by circuitry 200.

An expression for the un-distortion factor U is given by equation (7):

$$U = \frac{1}{D} = \frac{{m}_{x}}{{m}_{y}} \qquad ( 7 )$$

In other words, from equation (7), the ratio of the width:height magnifications, U, applied to the digitized images output from array 60, for display on screen 32, is the inverse of the ratio of the width:height magnifications, D, generated by optics 100. An example for applying the required magnifications to the digitized images from array 60 is described below.

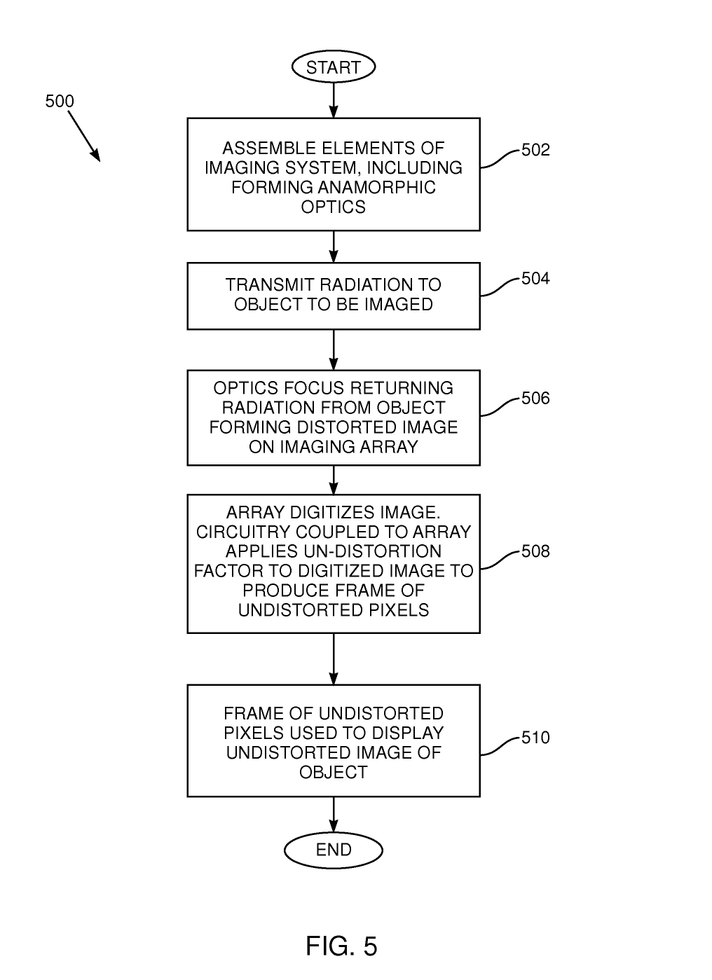

FIG. 5 is a flowchart 500 describing steps in operation of system 10, according to an embodiment of the present invention. The steps of flowchart 500 assume that camera 38 is being used, and those having ordinary skill in the art will be able to adapt the description, mutatis mutandis, for the case of camera 438.

In an initial step 502, the elements of system 10 are implemented, generally as described above with respect to FIG. 1. The implementation includes forming optics 100, and the optics are typically formed according to equations (1)-(6) and the associated descriptions, for a predetermined object distance from the optics, a predetermined aspect ratio of the object, and a predetermined aspect ratio and size of array 60. Optics 100 are assumed to be anamorphic, having a distortion factor D, as defined by equation (2).

In an irradiation step 504 radiation is irradiated from optical channels 46 into cavity 12. Typically, although not necessarily, the radiation comprises light in the visible spectrum. However, in some embodiments the radiation comprises non-visible components, such as infra-red and/or ultra-violet radiation.

The radiation illuminates objects within cavity 12, including walls of the cavity, and returning radiation from the illuminated optics is acquired by optics 100.

In an image acquisition step 506 optics 100 receive incoming radiation from the illuminated objects. The optics focus the acquired incoming radiation to an image of the illuminated objects, the image being formed on array 60. The focusing of the radiation is performed by the optics transmitting the acquired incoming radiation to array 60 via entrance face 86 of prism 80, hypotenuse face 82 of the prism, and exit face 84 of the prism.

In a digital image step 508 array 60 and circuitry 200 digitize the image focused onto array 60, to form a frame, or set, of pixels of the distorted image. The circuitry then applies an un-distortion factor U, defined above by equation (7) to the frame of pixels, to generate a set of pixels representative of an undistorted image. The application of un-distortion factor U typically involves addition of pixels, removal of pixels, and/or change of value of pixels of the digitized image received from array 60, so as to produce a frame of digitized pixels in an undistorted format. The following examples explain how pixels of an undistorted image are generated.

A first example assumes that optics 100 image object 130, with an aspect ratio of 4:3, onto array 60, and that array 60 corresponds to the array of the disclosed embodiment referred to above, having an aspect ratio of 200:112. The image from array 60 is then “undistorted” by circuitry 200 to be suitable for display on screen 32 as a 768 pixels wide×576 pixels high image, i.e., as an image having the same aspect ratio as object 130.

Optics 100 are configured to have a distortion factor D corresponding to the first numerical example above, i.e. the ratio of the width:height magnifications is 1.34.

Circuitry 200 “undistorts” the digitized image from array 60 by applying an un-distortion factor U, equal to

$$\frac{1}{1.34} = 0.75$$

from equation (7). This factor corresponds to the ratio of width:height magnifications introduced by circuitry 200 into the pixels received from array 60

In the y direction, array 60 generates 200 pixels, and screen 32 displays 768 pixels in this direction, for a width magnification of 3.84.

In the x (height) direction, array 60 generates 112 pixels, and screen 32 displays 576 pixels in this direction, for a height magnification of 5.14. The ratio of the width:height actual magnifications,

$$\frac{3.84}{5.14} ,$$

corresponds to the un-distortion factor U=0.75, introduced by circuitry 200.

A second example assumes that screen 32 has pixel dimensions of 1280×720, for an aspect ratio of 16:9. This aspect ratio substantially corresponds to the aspect ratio of array 60 (200:112). Thus an object with aspect ratio 16:9 may be imaged without distortion onto array 60, and there is no “undistortion” required in generating the 1280×720 pixels for screen 32. Since there is no distortion introduced by optics 100, the optics in this case may be spherical optics, or equivalent to spherical optics. In this second example the width magnification for screen 32 is

$$\frac{1280}{200} ,$$

and the height magnification is

$$\frac{720}{112} ,$$

both magnifications having the same value of approximately 6.4.

Consideration of the values above shows that for these examples circuitry 200 introduces pixels into the digitized values received from array 60, so as to produce a frame of pixels representative of an undistorted image. The introduction is typically by interpolation between the values from the array. Thus, in the y direction, circuitry 200 interpolates between the 200 values received to generate 768 pixels for the first example, or 1280 pixels for the second example, corresponding to the number of columns displayed by screen 32. Similarly, in the x direction, circuitry 200 interpolates between the 112 values received to generate 576 pixels for the first example, or 720 pixels for the second example, corresponding to the number of rows displayed by screen 32. The method of interpolation implemented by circuitry 200 may comprise any convenient interpolation method known in the art.

Those having ordinary skill in the art will be able to adapt the examples above to evaluate width and height magnifications introduced by circuitry 200 for other object aspect ratios, and for other array aspect ratios.

In a final display step 510, processor 20 receives from circuitry 200 a frame of pixels corresponding to an undistorted image of object 130, and displays the undistorted image on screen 32.

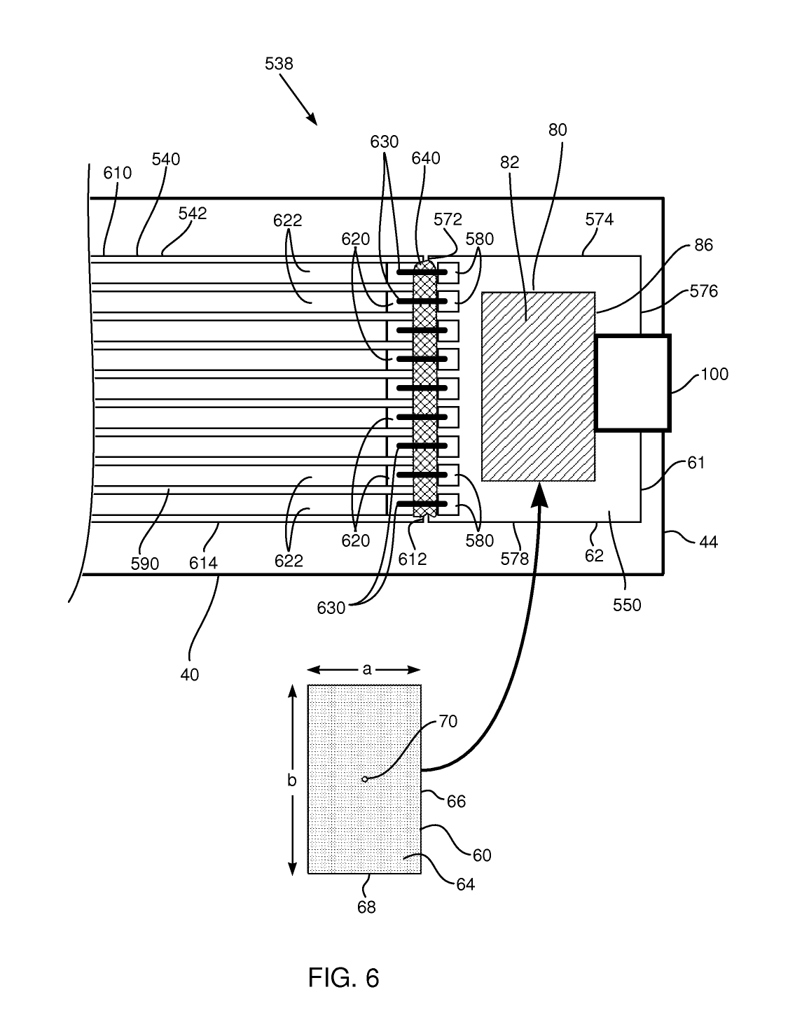

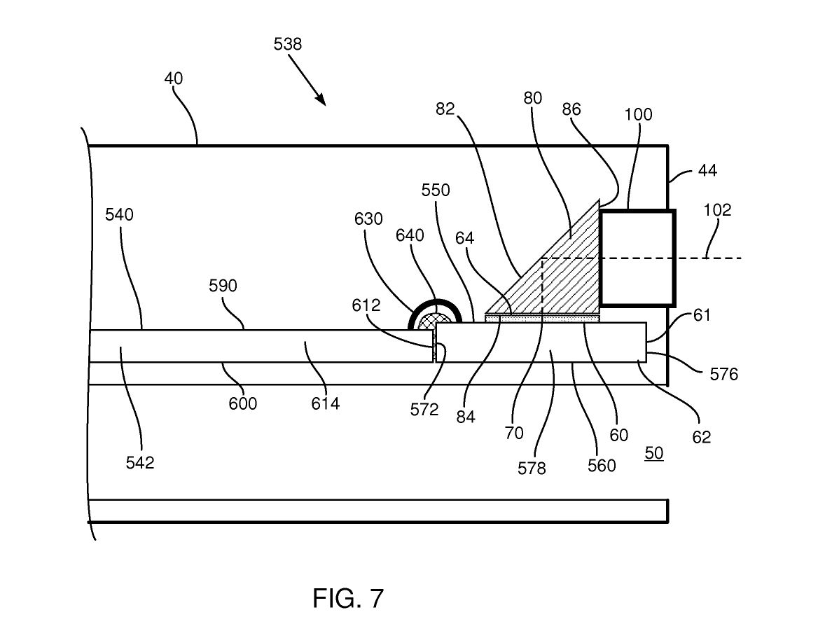

FIG. 6 is a schematic top view of a camera 538, and FIG. 7 is a schematic sectional view of the camera, according to an embodiment of the present invention. Apart from the differences described below, the operation of camera 538 is generally similar to that of camera 38 (FIGS. 2A-2E), and elements indicated by the same reference numerals in both cameras 38 and 538 are generally similar in construction and in operation. In contrast to camera 38, camera 538 does not have circuitry 200 connected to planar substrate 62; rather, a circuit 540, generally similar to flexible printed circuit board 240 and also referred to herein as flexible circuit 540 connects to the planar substrate. Flexible circuit 540 comprises a circuit dielectric substrate 542 which is connected directly to substrate 62.

In the following description, substrate 62 is assumed to be in a generally parallelepiped form, having an upper plane surface 550 upon which array 60 is formed, a lower plane surface 560, and four plane edges 572, 574, 576, and 578. (It will be understood that the references herein to upper and lower are with respect to the figures herein, and that camera 538 may be used in any orientation.) A plurality of substantially similar pads 580, that connect by conducting elements (not shown in the figure) to array 60, are mounted on surface 550.

In addition, while circuit 540 is flexible, in the circuit's “flattened” form dielectric substrate 542 may also be assumed to be in a generally parallelepiped form, having an upper plane surface 590, a lower plane surface 600, and plane edges 610, 612, and 614. Substrate 542 also has a further plane edge, not shown in the figure. A plurality of substantially similar pads 620, respectively connected to conducting traces 622, are formed on surface 590. The numbers of pads 580 and pads 620 correspond to each other. While FIG. 6 illustrates nine pads 580, pads 620, and related elements, it will be understood that this number is by way of example, and there may be fewer or more than nine pads 580, pads 620, and related elements in embodiments of the present invention.

In order to implement the connection between substrate 62 and circuit 540, array plane edge 572 of substrate 62 is butted to circuit plane edge 612 of circuit 540. The butting of the two edges is typically accomplished by placing the substrate and the circuit on a working surface (not shown in the figure) and sliding the two edges together. Once the two edges are butted together, pads 580 are connected to respective pads 620, in a one-to-one arrangement, by generally similar conducting connectors 630. The numbers of connectors 630 corresponds to the number of pads 580 (and the number of pads 620). The connection of connectors 630 to pads 580 and pads 620 may be by any method known in the art, such as, but not limited to, soldering and/or wire bonding.

A cement 640 may be applied to substrate 542 and substrate 62, between the butted edges as well as in a region close to the edges, so that when the cement hardens there is a fixed mechanical connection between the two substrates. Depending on the manufacturing procedure used to assemble camera 538, cement 640 may be applied before connectors 630 are connected between pads 580 and pads 620. Alternatively, cement 640 may be applied after connectors 630 are connected, in which case the cement may also be used to hold connectors 630 in position.

In a disclosed embodiment substrate 62 is approximately microns thick, and substrate 542 is approximately 50 microns thick. However, these thicknesses are by way of example, and both substrates may have thicknesses larger or smaller than the values noted here. It will be understood that plane surface 550 and plane surface 590 are parallel, and, depending on the thicknesses of substrate 62 and substrate 542, may be coplanar.

Signals driving array 60 may be transferred to the array via traces 622, and the traces may also be used to convey signals from the array to processor 20 (FIG. 1).

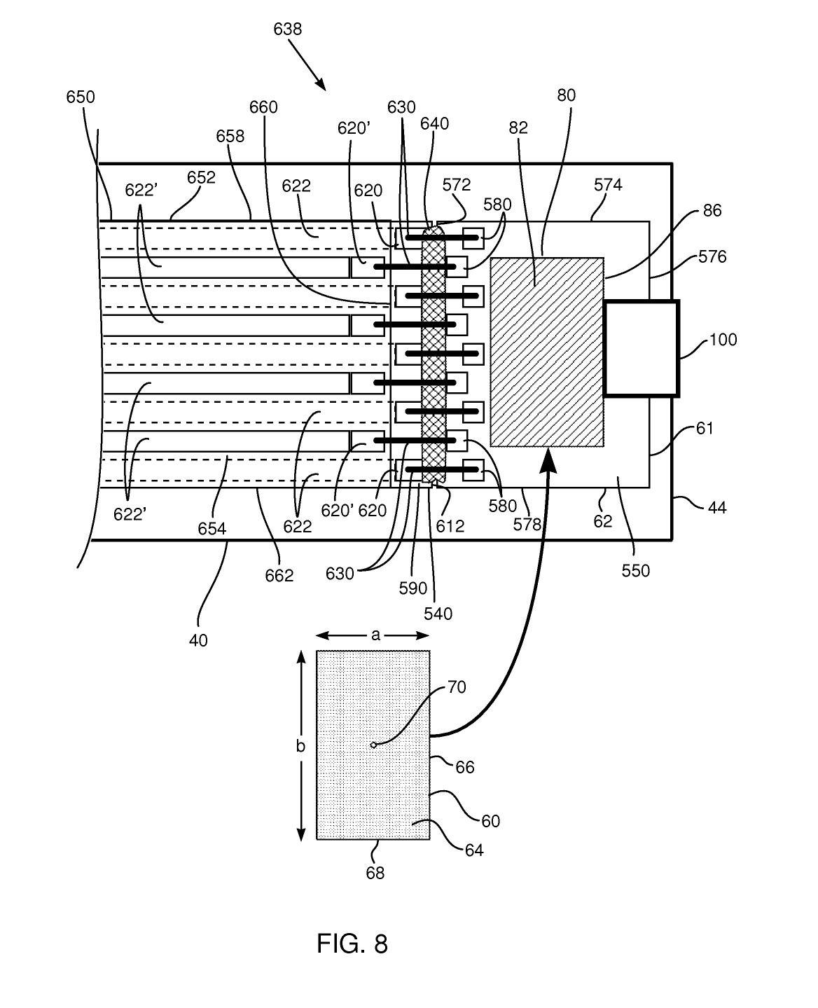

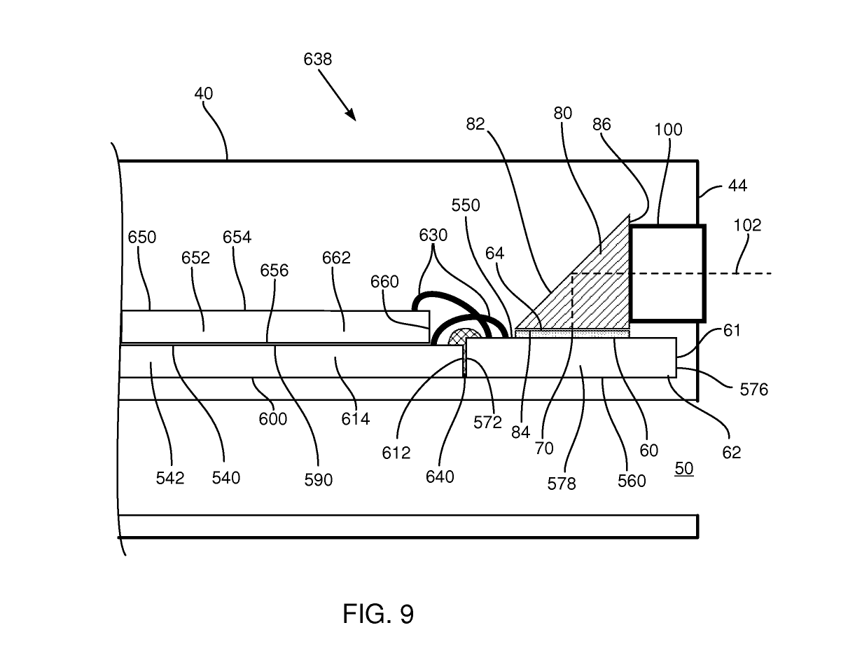

FIG. 8 is a schematic top view of a camera 638, and FIG. 9 is a schematic sectional view of the camera, according to an embodiment of the present invention. Apart from the differences described below, the operation of camera 638 is generally similar to that of camera 538 and elements indicated by the same reference numerals in both cameras 638 and 538 are generally similar in construction and in operation.

Camera 538 uses flexible circuit 540, and all traces 622 are formed on surface 590 of the substrate of the flexible circuit. In some cases, for example where circuit 540 is relatively narrow, forming all traces 622 on a single surface may be challenging from a manufacturing point of view, since the traces may need to be relatively narrow as well as being relatively close to each other. As is described below, camera 638 implements traces that may be wider than those of camera 538, as well as being able to be further apart from each other.

In camera 638 only some of traces 622, and their corresponding pads 620, are formed on surface 590 of circuit 540. By way of example, in the embodiment described herein five traces 622 and five pads 620 are formed on surface 590.

The five traces 622 on surface 590 are indicated by broken lines in the figure. In order to accommodate other traces and their respective pads, camera 638 uses a second flexible circuit 650, which may be located, as illustrated in the figures, above circuit 540. Circuit 650 is generally similar in form to circuit 540, being formed of a dielectric substrate 652 which in turn has an upper surface 654, a lower surface 656, and edges 658, 660, 662, shown in the figure, as well as one edge not shown. By way of example circuits 540 and 650 are assumed to have similar dimensions, but this is not a requirement of embodiments of the present invention, so that at least some of the dimensions of the two flexible circuits may be different. Traces and pads mounted on circuit 650 are distinguished from traces and pads mounted on circuit 540 by adding an apostrophe ′ to the identifying numeral for circuit 650. Thus, traces 622′ and respective pads 620′ (herein assumed to comprise four traces and four pads), that have not been formed on surface 590, are formed on surface 654 of substrate 652.

Circuit 650 is mounted above circuit 540 so that all pads 620, 620′ are accessible, i.e., so that after mounting the pads are able to be used for connecting to connectors 630. The accessibility to pads 620 may be achieved by offsetting edge 660 to be “further back” from edge 572 than is edge 612 of substrate 542 (as is stated above, edge 572 and edge 612 are butted together). In other words, there is a preset distance between edge 660 and edge 572, the preset distance being equal to, or greater than, the length of pads 620. The mounting of circuit 650 by the preset distance, in the “further back” manner, forms pads 620 and 620′ into a staggered arrangement that is visible in FIG. 8. In some embodiments, pads 580 (for array 60) may be staggered so as to correspond to the staggering of pads 620, as is illustrated in the figure. However, staggering of pads 580 is not a necessary requirement of embodiments of the present invention, so that in some embodiments pads 580 are not staggered.

The connection between pads 580 of substrate 62 and pads 620, 620′ on circuits 540, 650 is performed in a generally similar manner to the connection between substrate 62 and circuit 540, described above with reference to camera 538. For example, edge 612 of circuit 540 is first butted to edge 572 of substrate 62. Circuit 650 is then fixedly attached to circuit 540 so that edge 660 is parallel to, and is at the preset distance referred to above, edge 572. The attachment may include cementing the two circuits together. Pads 580 are then connected to pads 620, 620′ in a one-to-one arrangement, by conducting connectors 630, as described above for camera 538.

As for camera 538, cement 640 may be applied to substrate 542 and substrate 62, between the butted edges as well as in a region close to the edges. If the cement is applied after connectors 630 have been connected, the cement may be used to hold the connectors to both circuit 540 and circuit 650 in place.

As is apparent from the figures for camera 638, using two flexible circuits mounted above each other, to connect to substrate 542, allows for greater width of the traces on the circuits, and for greater separation of the traces. Offsetting the circuits allows access to all pads on the circuits.

It will be appreciated that there may be more than two flexible circuits mounted above each other. Thus, embodiments of the present invention include cases where more than two flexible circuits are mounted above each other, with appropriate offsets between the circuits to allow access to all pads of the circuits.

It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. Apparatus, comprising:
an integrated circuit (IC) die, comprising:
a semiconductor substrate having a substrate plane face and a substrate edge;
an imaging array formed on the substrate plane face; and
a plurality of array pads mounted on the substrate plane face and connected to the imaging array;
a circuit, comprising:
a circuit substrate having a circuit face and a circuit edge butted to the substrate edge;
a plurality of circuit pads mounted on the circuit face; and
a plurality of traces mounted on the circuit face and connected to the circuit pads; and
a plurality of connectors coupling the array pads to the circuit pads.

2. The apparatus according to claim 1, wherein the circuit comprises a flexible circuit.

3. The apparatus according to claim 1, wherein the circuit substrate comprises a dielectric.

4. The apparatus according to claim 1, and comprising cement fixedly holding the circuit edge to the substrate plane edge.

5. The apparatus according to claim 4, wherein the cement holds the connectors in place.

6. The apparatus according to claim 1, wherein the IC die comprises a multiplicity of additional array pads mounted on the substrate plane face and connected to the imaging array, the apparatus further comprising:
an additional circuit, comprising:
an additional circuit substrate having an additional circuit face and an additional circuit edge separated from the substrate plane edge by a preset distance;
a multiplicity of additional circuit pads mounted on the additional circuit face; and
a multiplicity of additional traces mounted on the additional circuit face and connected to the additional circuit pads; and
a multiplicity of additional connectors coupling the additional array pads to the additional circuit pads.

7. The apparatus according to claim 6, wherein the preset distance is greater than or equal to a length of the circuit pads.

8. The apparatus according to claim 6, wherein the additional circuit is fixedly attached to the circuit.

9. The apparatus according to claim 1, wherein the IC die, the circuit, and the plurality of connectors are located in a distal end of an endoscope.

10. An endoscope camera, comprising:
a cylindrical enclosure having an enclosure diameter;
an integrated circuit (IC) die mounted within the enclosure, the die comprising:
a semiconductor substrate having a substrate plane face and a substrate edge;
an imaging array formed on the substrate plane face; and
a plurality of array pads mounted on the substrate plane face and connected to the imaging array;
a right-angle transparent prism having a rectangular entrance face, an exit face, and an hypotenuse configured to reflect radiation from the entrance face to the exit face, the entrance face having a first edge longer than a second edge, the prism being mounted within the enclosure so that the first edge is parallel to the enclosure diameter and so that the exit face mates with the substrate plane face;
a circuit, comprising:
a circuit substrate having a circuit face and a circuit edge butted to the substrate edge;
a plurality of circuit pads mounted on the circuit face; and
a plurality of traces mounted on the circuit face and connected to the circuit pads;
a plurality of connectors coupling the array pads to the circuit pads; and
optics, configured to receive incoming radiation from an object, mounted so as to transmit the incoming radiation to the imaging array via the entrance and exit faces of the prism.

11. A method, comprising:
providing an integrated circuit (IC) die comprising a semiconductor substrate having a substrate plane face and a substrate edge;
forming an imaging array on the substrate plane face;
mounting a plurality of array pads on the substrate plane face and connecting the pads to the imaging array;
providing a circuit comprising a circuit substrate having a circuit face and a circuit edge;
butting the circuit edge to the substrate edge;
mounting a plurality of circuit pads on the circuit face;
mounting a plurality of traces on the circuit face and connecting the traces to the circuit pads; and
coupling a plurality of connectors between the array pads and the circuit pads.

12. The method according to claim 11, wherein the circuit comprises a flexible circuit.

13. The method according to claim 11, wherein the circuit substrate comprises a dielectric.

14. The method according to claim 11, and comprising fixedly cementing with cement the circuit edge to the substrate plane edge.

15. The method according to claim 14, wherein the cement holds the connectors in place.

16. The method according to claim 11, wherein the IC die comprises a multiplicity of additional array pads mounted on the substrate plane face and connected to the imaging array, the method further comprising:
providing an additional circuit, comprising:
an additional circuit substrate having an additional circuit face and an additional circuit edge separated from the substrate plane edge by a preset distance;
a multiplicity of additional circuit pads mounted on the additional circuit face; and
a multiplicity of additional traces mounted on the additional circuit face and connected to the additional circuit pads; and
coupling a multiplicity of additional connectors between the additional array pads and the additional circuit pads.

17. The method according to claim 16, wherein the preset distance is greater than or equal to a length of the circuit pads.

18. The method according to claim 16, and comprising fixedly attaching the additional circuit to the circuit.

19. The method according to claim 11, wherein the IC die, the circuit, and the plurality of connectors are located in a distal end of an endoscope.
